(19) United States
(12) Patent Application Publication
(10) Pub. No.: US 2024/0170151 A1
(43) Pub. Date: May 23, 2024

(54) INTERFACE AND DEEP LEARNING MODEL FOR LESION ANNOTATION, MEASUREMENT, AND PHENOTYPE-DRIVEN EARLY DIAGNOSIS (AMPD)

(71) Applicant: Amrit.ai Inc. d/b/a Picture Health, Cleveland, OH (US)

(72) Inventor: Nathaniel M. Braman, Cleveland, OH (US)

(21) Appl. No.: 18/512,758

(22) Filed: Nov. 17, 2023

Related U.S. Application Data
(60) Provisional application No. 63/426,098, filed on Nov. 17, 2022.

(51) Int. Cl. G16H 50/20; G06T 7/00; G06T 7/11; G16H 30/40

(52) U.S. Cl. CPC G16H 50/20; G06T 7/0012; G06T 7/11; G16H 30/40; G06T 2207/20084; G06T 2207/30096

(57) ABSTRACT

Methods and systems that provide annotation, measurement, phenotyping, diagnosis (AMPD), and other medical predictions from a medical image are disclosed. In these systems, a medical image may be presented in an interface, such as a graphical user interface. When a trained person clicks on a point or indicates a region, a machine learning segmentation model segments the area around the point or region to identify one or more lesions. Machine models are used to establish the measurements, phenotypical characteristics, and diagnosis or other medical predictions. Those machine models may be trained to use the measurements, the phenotypical characteristics, or image features descriptive of the lesion in making the medical predictions. The image features descriptive of the lesion may be deep features extracted from a deep learning segmentation model, or they may be radiomic features.

### CROSS-REFERENCE TO RELATED APPLICATIONS

This application claims priority to, and the benefit of, U.S. Provisional Patent Application No. 63/426,098, filed Nov. 17, 2022. The contents of that application are incorporated by reference herein in their entirety.

### TECHNICAL FIELD

The disclosed technology pertains to a system and interface for early diagnosis of cancer and other pathologies based upon medical images.

### BACKGROUND

Conventional approaches to diagnosing cancer and other diseases based on medical imaging may utilize combinations of manual and automated steps and are often performed across multiple distinct systems and health information environments, and with multiple users providing review, confirmation, annotation, and other inputs.

The varied approaches, systems, interfaces, and participants in such processes often result in lengthy wait times for output of results, inaccurate or incomplete results, and various inefficiencies. As an example, some diagnostic systems may automatically identify a lesion depicted by a medical image, and may perform various additional tasks based on such an identification (e.g., resource intensive additional analysis, image annotation and markup, distribution of images and diagnoses to clinicians or others associated with the patient). Such processes are often queue-driven and performed across multiple local and remote devices within a single information system, such that a user providing care or support to a patient at the point-of-care (“POC”) will often have limited or no visibility on the status of such tasks, and little ability to influence or intervene in the outcome if needed. Such processes are often also performed across information systems from multiple different parties, with images, information, and other input being passed to third parties via communication interfaces that provide minimal feedback or interactivity.

While timely and high-quality care is certainly possible with a workflow having such limitations, it is not uncommon to have breakdowns in the workflow that are difficult or impossible to address, and that can contribute to delays, inefficiencies, inaccuracies, and poor patient outcome. As an example, where an automatic diagnosis performed early in the workflow erroneously segments or identifies a lesion, a number of additional steps and actions may be taken before the error is identified and corrected. In the meantime, the patient, clinician, and others involved in the patient care may have been waiting for results of a workflow that now needs to be re-performed, or may have begun to take action or provide care based on workflow output that is now in question.

What is needed, therefore, is an improved system and interface for early diagnosis of cancer and other pathologies.

### BRIEF SUMMARY

Aspects of the invention relate to methods and systems for establishing annotation, measurement, phenotypical characteristics, and diagnosis of, or other medical predictions concerning, a lesion on a medical image.

In a method according to one aspect of the invention, one or more measurements and one or more phenotypical characteristics of a lesion are established using a first machine learning model operating on a machine segmentation of a lesion indicated in a medical image. Using the first machine learning model or a second machine learning model, a medical prediction concerning the lesion is provided using one or more of at least some of the measurements, at least some of the phenotypical characteristics, or features extracted from the machine segmentation.

In a practical embodiment of the method described above, a trained person would click on or otherwise indicate a lesion or a region containing a lesion using a graphical user interface (GUI). A machine segmentation model would produce a machine segmentation of the lesion using the indication from the GUI. The system would proceed to produce measurements and define phenotypical characteristics of the lesion automatically and would provide the medical prediction. The phenotypical characteristics would typically be clinically-known phenotypical characteristics typically used by trained persons to make clinical judgments and predictions about lesions of that particular type. The medical prediction could be a diagnosis (e.g., benign, malignant, or the particular type of lesion), a prediction concerning response to a particular treatment, a prediction concerning survival, a prediction concerning progression, etc.

Another aspect of the invention relate to systems implementing these methods. These systems would generally include a processor, such as a microprocessor, and memory, and would establish, e.g., the GUI used to obtain the indication of interest and provide the output. Systems according to embodiments of the invention may be physically distributed and networked with one another, or they may be physically located at the same location.

Other aspects, features, and advantages of the invention will be set forth in the description that follows.

### BRIEF DESCRIPTION OF THE DRAWING FIGURES

The invention will be described with respect to the following drawing figures, in which like numerals represent like features throughout the description, and in which:

FIG. 1 is a high-level flow diagram of a method that uses a trained machine model or models to perform annotation, measurement, phenotyping, and diagnosis of a lesion in a medical image;

FIG. 2 is a schematic diagram of a system for performing the method of FIG. 1;

FIGS. 3A-3D are schematic illustrations of a graphical user interface (GUI) that presents a medical image along with annotation, measurement, phenotyping, and diagnosis or risk information;

FIGS. 4A-4D are schematic illustrations of another type of GUI that presents a medical image along with annotation, measurement, phenotyping, and diagnosis or risk information;

FIG. 5 is a graph illustrating the consensus between manual diameter measurements of lesions by trained persons with estimated lesion diameter measurements made by an example system according to an embodiment of the invention;

FIG. 6 is a set of graphs illustrating the consensus between manual measurements of nine phenotypical characteristics of lesions with calculated measurements for those phenotypical characteristics made by an example system according to an embodiment of the invention; and

FIG. 7 is a graph illustrating receiver-operating curves (ROCs) for lesion morphology stratification by the example system.

### DETAILED DESCRIPTION

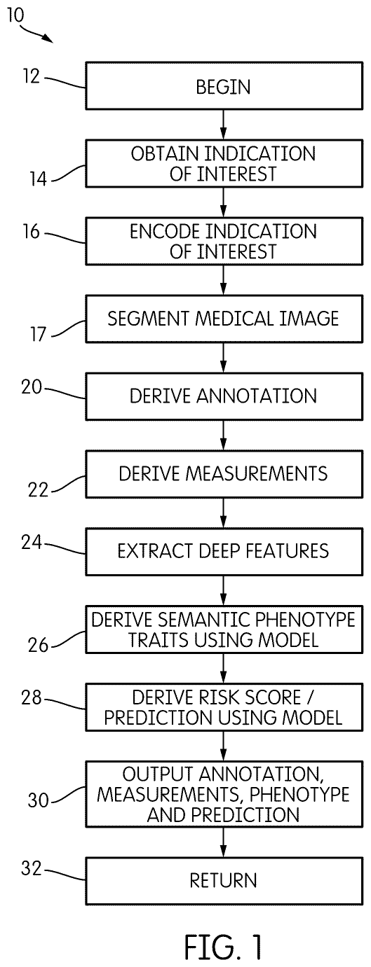

FIG. 1 is a high-level flow diagram of a method, generally indicated at 10, that uses a trained machine model or models to perform a series of tasks including annotation, measurement, phenotyping, and diagnosis given a medical image that includes one or more lesions. As will be described below in more detail, portions of this description may refer to method 10 and to systems that implement it as “semi-automated” in that method 10 performs all of these tasks based on a single input from a user, typically a radiologist, oncologist, or another such trained person.

Here, the term “medical image” refers to any kind of medical imagery, including computed tomography (CT) scans, magnetic resonance imaging (MRI) scans, positron emission tomography (PET) scans, and X-ray images. The term “medical image” also applies to applications of these types of modalities to specific body parts, as in the case of mammography and digital breast tomosynthesis. As those of skill in the art will understand, a medical image need not be the result of a single exposure or capture event. Rather, a single medical image may be a reconstruction or interpolation from a larger image dataset, e.g., a particular plane or “slice” from a helical CT scan. Moreover, the medical image used in method 10 need not necessarily be two-dimensional: in many cases, the medical image may be a three-dimensional image showing, e.g., some compartment or interior volume of the body, although two-dimensional “slices” or projections of that three-dimensional image may be used in particular tasks and for particular purposes.

The trained person may access a single medical image or a series or set of related medical images from a scan or study. Typically, “access” occurs by retrieving one or more medical images from a database or other storage medium and displaying them on a desktop computer, a tablet computer, a touchscreen display, etc. In clinical use, the database may be a Picture Archiving and Communication System (PACS) database; research and other non-clinical applications of method 10 may use a PACS database or a database or repository of some other sort.

As was noted above, the medical image includes one or more lesions. Here, the term “lesion” is used in the general sense to indicate any kind of injury to, or disease in, an organ or tissue that is discernible in a medical image, e.g., a lung nodule visible on a CT image. Any lesion may be either benign or malignant (e.g., a cancerous tumor). A trained person may do any number of things with such a medical image. For example, a radiologist might “read” a scan in traditional fashion and produce a radiology report.

A traditional radiology report might include information on the precise boundaries of any lesions, which this description refers to as annotation or annotations. A traditional report might also include clinical measurements of any lesions; an indication of characteristics related to the biology or appearance of the lesion, which this description refers to as the phenotype of a lesion; and an indication of a diagnosis, such as whether a particular lesion is benign or malignant.

Artificial intelligence (AI) predictive systems used in medicine can easily become “black boxes” that offer diagnoses, treatment plans, or predictions regarding a lesion with no overt reasoning or evidence provided to support whatever diagnoses, treatment plans, or other predictions that are offered. However, as will be described below in more detail, method 10 and systems that implement it can provide both medical predictions and information derived from those predictions, like treatment plans, alongside the same sort of information that would be found in a traditional radiology report, thus giving trained professionals a basis on which to evaluate any clinical prediction or recommendation that might be made.

Method 10 begins at task 12 and continues with task 14, in which an indication of interest is obtained from the trained person. In task 14 of method 10, the trained person indicates one or more lesions or other regions of concern in the medical image or series of medical images. The method by which this is done is not critical. For example, the trained person may click on the lesion in a graphical user interface using a mouse or trackpad, tap on the lesion if a device with a touchscreen is being used, circle the lesion or mark it in some other way with a stylus, etc. While the method by which the trained person indicates lesions in the medical image is not critical, it is advantageous if that method is quick and convenient, because method 10 preferably requires as little of the trained person's time as possible.

The location or locations at which the trained person clicked or otherwise made an indication are recorded as the indication of interest. An indication of interest may comprise multiple points from multiple lesions or regions. The indication of interest essentially describes structures or regions that are of concern to the trained person and will be used to make one or more medical predictions in later tasks of method 10. In some cases, once the indication of interest is obtained, it may be displayed or overlaid on the medical image so that the trained person can confirm that the input was correctly received, but that need not be the case in all embodiments.

Typically, the indication of interest is stored numerically as a set of two-dimensional or three-dimensional coordinates. Those coordinates may be expressed in any useful frame of reference, e.g., relative to the medical image itself (i.e., the pixel or voxel coordinates of the indication of interest in the medical image), relative to an organ or anatomical feature in the medical image, or relative to some other point of origin. If method 10 is operating on a set of medical images, the indication of interest would typically also include an indication of the image to which it corresponds.

With respect to method 10 of FIG. 1, once the indication of interest has been obtained, method 10 proceeds with task 16. In task 16, method 10 encodes the indication of interest in such a way that it can be used by a segmentation model, a machine model that is specialized for and trained in the task of identifying the structures in an image. In other words, the indication of interest is processed and stored in a form that can be input or integrated into a segmentation model. How that is done will depend on several factors, including the input requirements of the segmentation model and the precise nature of the indication of interest. Some segmentation models may be able to use the two- or three-dimensional coordinates of the indication of interest as an input without further encoding or modification, although it may be necessary to translate the coordinates into a different frame of reference in some cases.

However, a set of coordinates alone may not be a suitable input for some segmentation models, and the indication of interest may be expressed in any number of ways. For example, an image-like matrix could be constructed that weights each pixel in accordance with metrics relevant to the likelihood that that pixel is a part of the lesion to which the indication of interest pertains, such as the Euclidean distance from the point or points that were clicked, or some other metric.

Segmentation models often use neural networks, such as convolutional neural networks (CNNs) and vision transformers, and those neural networks usually require input in the form of an image. In those cases, a distance map could be encoded as an additional image channel or layer, or as a separate image. Other types of segmentation models that do not rely on neural networks could also be used, including active contour and region-growing approaches.

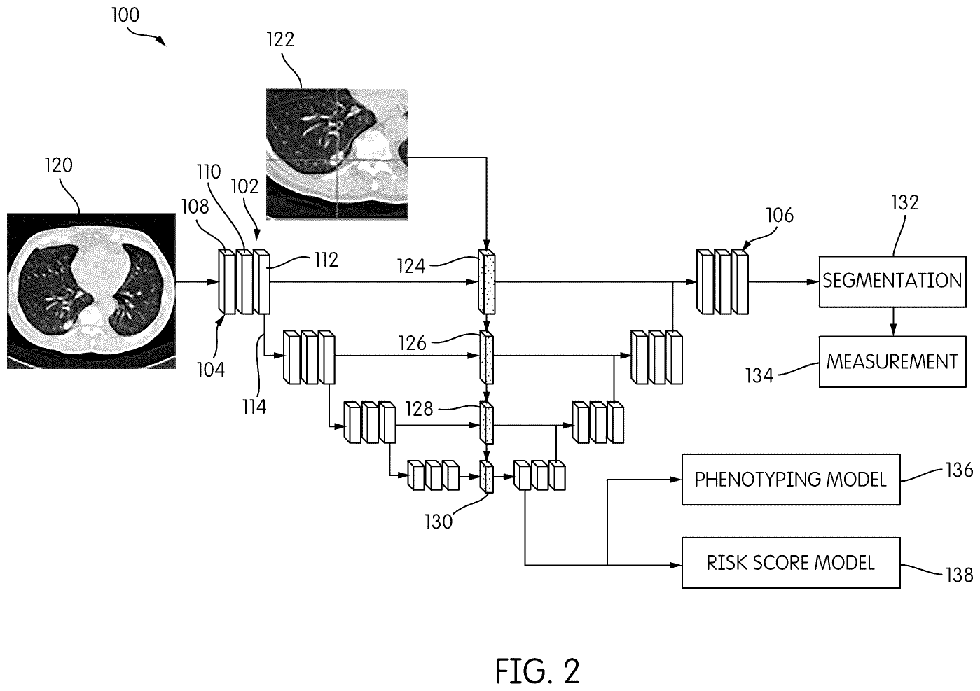

In the illustrated embodiment, method 10 uses a U-net, a CNN architecture that is particularly suited for image segmentation tasks. FIG. 2 is a schematic diagram of a system 100 that could be used to implement method 10. The U-net 102 used in system 100 includes, as is typical, a contracting path 104 and an expansive path 106. Typically, as is known in the art, each layer of the contracting path 104 would include successive convolutions 108, 110 followed by a rectified linear unit 112 and max-pooling operation 114. In the expansive path 106, the feature map from the corresponding layer of the contracting path 104 is cropped and concatenated onto the upscaled feature map.

As shown in FIG. 2, the medical image 120 is input to the contracting path 104 of the U-net 102. Relevant to task 14 of method 10, the indication of interest 122 is input to attention modules 124, 126, 128, 130 that are interposed between corresponding contracting and expansive layers of the the U-net 102. In this particular example, the indication of interest 122 may be input to the attention modules 124, 126, 128, 130 as an image distance map, centered around the point that was clicked or otherwise indicated in task 14 of method 10. The distance map may have a gradient that is more intense at and around the point of interest and less intense radiating outward from it. Reverse gradients may also be used. As was noted above, distance maps may be based on Euclidean distance, or on other distance measures, like Manhattan distance and Chebyshev distance. If more than one lesion was indicated in task 14, the resulting distance map image may have multiple focal points with a gradient around each of the focal points.

With respect to FIG. 1, method 10 continues with task 18 and the medical image 120 is segmented, with an image segmentation 132 output by the expansive path 106. In this embodiment, the image segmentation 132 can be assumed to be a two-dimensional segmentation that delineates the boundaries of a lesion relative to a single medical image. In other embodiments, the segmentation may be three-dimensional. A single segmentation may also involve identifying the lesion on multiple medical images acquired at a single scanning timepoint, or segmentation of the lesion on multiple medical images acquired at different scanning timepoints.

As indicated in FIG. 1, once segmentation is performed in task 18, method 10 continues with task 20 and an annotation is derived. This may be done in any number of ways. In the simplest embodiment, the output of task 18, the segmentation, may be used directly as an annotation. However, in other embodiments, it may be necessary to alter or adapt the segmentation to serve as a clinical annotation, e.g., by changing coordinate systems into a clinically-relevant coordinate system, outputting the segmentation in a particular format, etc. Task 20 of FIG. 1 is meant to encompass all of the steps that may be necessary in converting or presenting a machine segmentation as a clinical annotation.

Once the segmentation is established in task 18, clinical measurements may be taken, as shown in task 22 of method 10. For convenience in illustration and explanation, FIG. 2 shows that a separate software module, measurement module 134, receives the segmentation data from the segmentation module 132 and makes measurements. However, the precise way in which these functions are divided between software modules and/or functions is not critical.

In general, measurement may include automatic computation of a straightforward clinical measure of lesion diameter, as well as short axis, area, volume, basic shape attributes, and other measurements. Measurement may also include the computation and/or extraction of complex measurements or features that cannot be calculated manually. This type of operation is referred to is radiomics.

In the field of radiomics, large amounts of quantitative data are extracted from medical images, such as CT, MRI, and PET scans, as well as classic X-ray images. This quantitative data is in the form of tens, hundreds, or even thousands of individual quantitative image features. Some of those features, like the size and shape of a lesion, are straightforward and would be understandable to any clinician. Other features, for example, relating to the texture of the image in and around the lesion, are less interpretable, or uninterpretable, to human eyes. The radiomic features are used with machine learning techniques to make medical predictions.

Examples of features that may be extracted and used include histogram features, textural features, filter- and transform-based features, and size- and shape-based features, including vessel features. As will be described below in more detail, vessel features can be considered to be a special case of size- and shape-based features. The classification of various radiomic features may vary depending on the authority one consults; the categories used here should not be considered a limitation on the range of features that could potentially be used.

Histogram features use the global or local gray-level histogram, and include gray-level mean, maximum, minimum, variance, skewness, kurtosis, etc. Measures of energy and entropy may also be taken as histogram or first-order statistical features. Texture features explore the relationship between voxels, the gray-level cooccurrence matrix (GLCM), the gray-level run-length matrix (GLRLM), gray-level size zone matrix (GLSZM), and gray-level distance zone matrix (GLDZM). Co-occurrence of local anisotropic gradient orientations (COLLAGE) features are another form of texture feature that may be used. (See P. Prasanna et al., “Co-occurrence of local anisotropic gradient orientations (collage): distinguishing tumor confounders and molecular subtypes on MRI,” in Int'l Conf. on Med. Image Computing and Computer-Assisted Intervention, pp. 73-80 (Springer, 2014).) Filter- and transform-based features include Gabor features, a form of wavelet transform, and Laws features.

Vessel features, i.e., features of the blood vessels in the peri-lesional region, may be used, including measures and statistics descriptive of vessel curvature and vessel tortuosity. (Sec, c.g., Braman, N., et al., “Novel Radiomic Measurements of Tumor-Associated Vasculature Morphology on Clinical Imaging as a Biomarker of Treatment Response in Multiple Cancers.” Clin. Cancer Res. 28 (20), pp. 4410-4424, (October, 2022).) Transform-based approaches to characterizing vessel features like curvature and tortuosity may also be used, such as Vascular Network Organization via Hough Transform (VaNgOGH). (Sec, c.g., Braman, N. et al. “Vascular Network Organization via Hough Transform (VaNgOGH): A Novel Radiomic Biomarker for Diagnosis and Treatment Response” in Medical Image Computing and Computer Assisted Intervention—MICCAI 2018 (eds. Frangi, A.F., et al.), pp. 803-811 (Springer, 2018)). As noted above, vessel features can be considered to be a special case of size- and shape-based features, considering the size and shape of the vessels, rather than the lesion. If vessel features are to be used, a segmentation of the vessels around the lesion may be performed in task 18 of method 10 to identify the vessels. Additional steps may also be taken, like the use of a fast-march algorithm to identify the centerlines of the vessels and steps to connect disconnected vessel portions, before extracting features from the vessels.

Once measurements are derived and/or extracted from the segmented image in task 22, method 10 continues with task 24. As was indicated above, radiomic features of the segmented lesion may be specifically extracted and used to make medical predictions. However, in many embodiments, it may not be necessary to perform a traditional radiomic feature extraction. Instead, post-attention deep features extracted from the U-net 102 may be sent to a machine learning model to make medical predictions, to establish semantic phenotype characteristics for a lesion, and for other purposes. This is the purpose of task 24 of method 10.

The “deep features” extracted in task 24 are essentially compressed, filtered versions of the medical image that have been through multiple trained convolution and downsampling operations, and thus have a reduced dimensionality. In the schematic of FIG. 2, deep features are extracted from the beginning of the expansive path 106, after the attention module 130.

Method 10 continues with task 26, in which semantic phenotype traits are derived using a machine-learning model. As shown in FIG. 2, the deep features extracted in task 24 are fed to a machine learning phenotyping model 136. The machine learning phenotyping model 136 is trained to derive a score or weight for any number of phenotypical characteristics of a lesion. The phenotypical characteristics that are derived would typically align with characteristics that a radiologist or other trained person would appreciate or note for that lesion. Examples include subtlety, structure, calcification, sphericity, margin, lobulation, spiculation, and texture. The output from the model 136 for each of these characteristics may be a score that is indicative of each trait. For example, a high score for sphericity might indicate that a particular lesion is strongly or mostly spherical, while a low score might indicate that a particular lesion is not spherical or is only weakly spherical. A high score for margins might indicate that a particular lesion has clearly defined margins, while a low score might indicate that it does not. However, the “sense” of the score or ranking provided by the machine learning model 136 may be normed among the different phenotypical characteristics, such that a high score is always indicative of a more problematic phenotype and a low score is always indicative of a less problematic phenotype, or vice-versa. If the phenotypical characteristic is generally appreciated in clinical practice, then the score will typically follow whatever clinical convention is used for that characteristic. Depending on the nature of the phenotypical characteristic, a score may also be a measurement of that characteristic.

In general, the information should be presented in a way that would be immediately understood by a trained person, and where an established clinical scale or metric for a particular trait is commonly used, the information may be presented using that established clinical scale or metric. The semantic phenotypical characteristics that are established by the machine learning model 136 may vary from embodiment to embodiment, and may be any characteristics that might be considered by a trained person, or any consistent characteristic that can be established and presented by a machine learning model 136.

In this embodiment, the machine learning phenotyping model 136 is a machine learning classifier. Any type of classifier may be used, depending on the nature of the medical prediction that is to be made, the nature of the features, and other factors. The classifier may be, e.g., a logistic regression or Cox proportional hazards model, a linear discriminant analysis (LDA) classifier, a quadratic discriminant analysis (QDA) classifier, a bagging classifier, a random forest classifier, a support vector machine (SVM) classifier, a Bayesian classifier, a Least Absolute Shrinkage and Selection Operator (LASSO) classifier, etc. A trained neural network may also serve as a classifier.

In this embodiment, the classifier is trained to make predictions (i.c., establish phenotype scores) using the deep features extracted from the U-net 102. However, the machine learning model 136 may be trained to make predictions based on any combination of features. For example, if radiomic features are extracted in task 22 of method 10, the machine learning model 136 may be trained to use some combination of deep features derived from the image segmentation process and radiomic features that are separately extracted from the segmented medical image.

As shown in FIG. 1, once phenotype characteristics have been established in task 26, method 10 continues with task 28, and a risk score or medical prediction is established in task 30. For purposes of this description, a “medical prediction” is anything medically relevant that can be predicted by method 10 and other methods like it. Medical predictions may include, but are not limited to, the diagnosis of a disease or the classification of a lesion according to its phenotype or genotype; prognoses and predictions of disease progression; predictions of whether a particular lesion is likely to respond to a particular treatment; predictions of whether the apparent growth of a lesion during treatment represents a true progression of the underlying disease or a pseudo-progression caused by treatment; predictions of whether a particular patient is likely to experience a particular side effect, like hyper-progression, from a particular treatment; and the like.

The risk score or medical prediction established in task 30 may be made by the same machine learning model 136 used to establish the phenotype characteristics, or it may be made by another machine learning model. That machine learning model may use any combination of deep features extracted from the segmentation process, radiomic features extracted from the medical image, general clinical or demographic information on the patient, or any other available information to make a prediction. In FIG. 2, a risk score/predictive model 138 takes deep features from the U-net 102, and in some cases, at least some of the outputs of the phenotyping classifier 136, as features to make a medical prediction. In the illustrated embodiment, the medical prediction is an overall risk score.

Once all of the phenotype characteristics and metrics have been calculated, method 10 proceeds with task 30, and the annotation, measurements, phenotype, and medical prediction or predictions are output. This may be done in any number of ways. For example, in some embodiments, the output may be a written or printed output, or a textual report that is output to the trained person or, if system 100 and method 10 are being used clinically, stored in an electronic medical record (EMR) system for the particular patient.

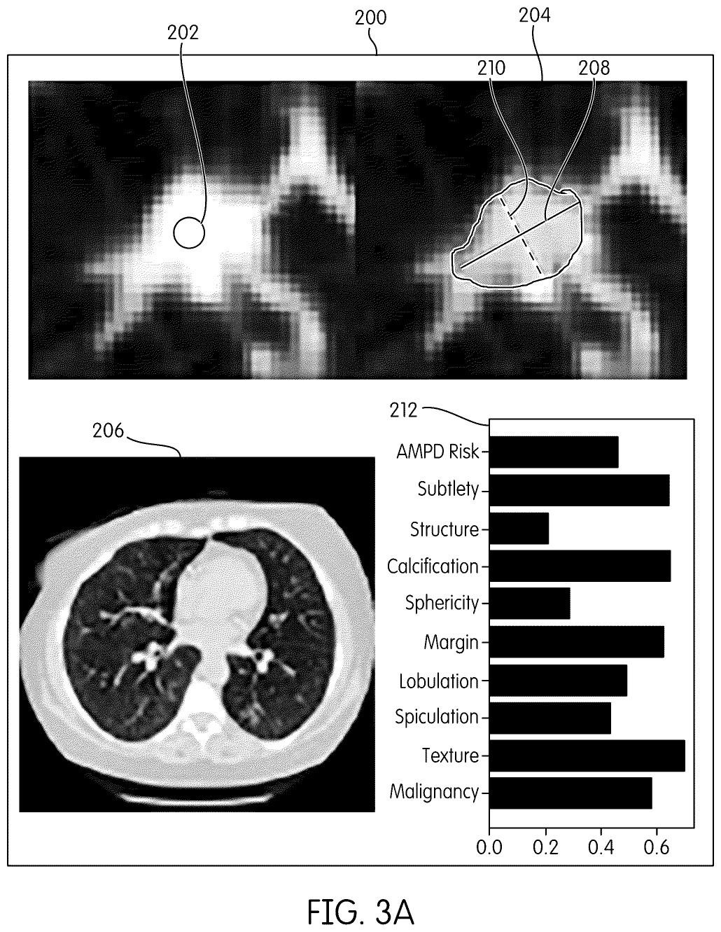

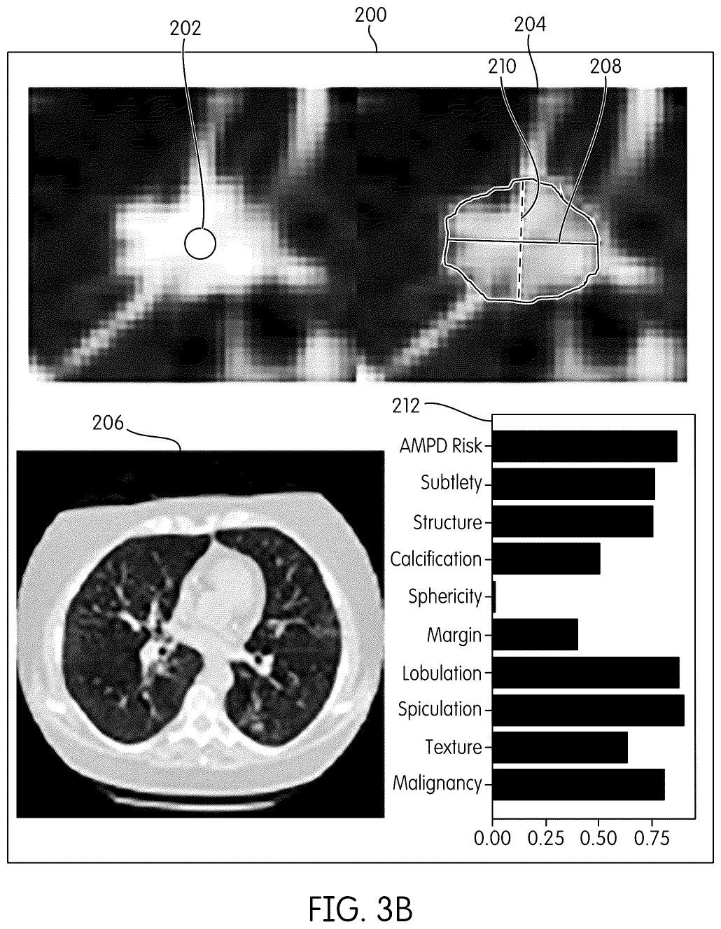

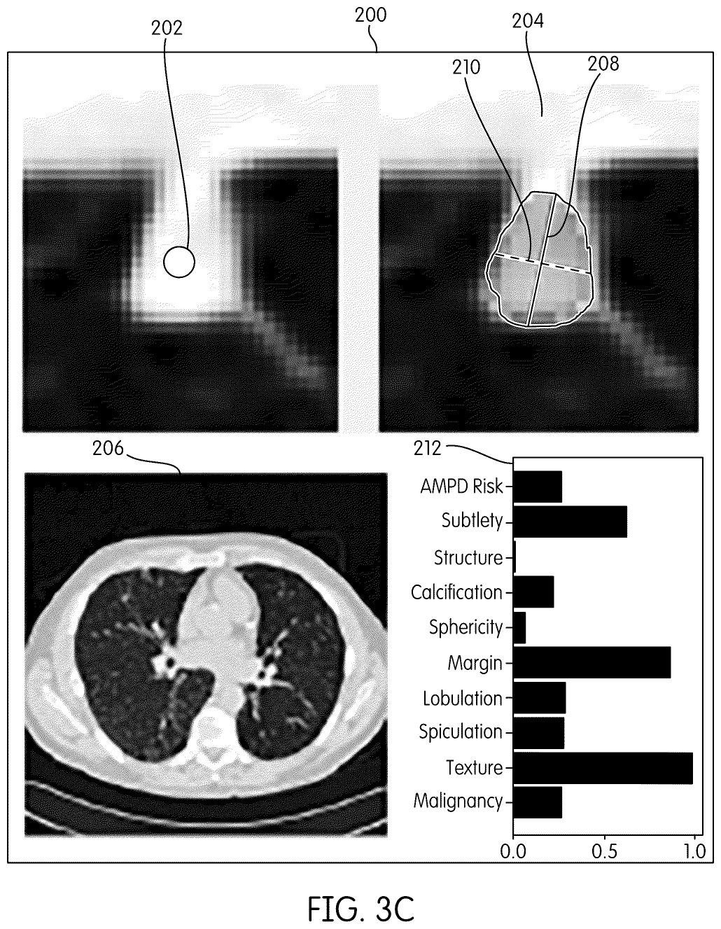

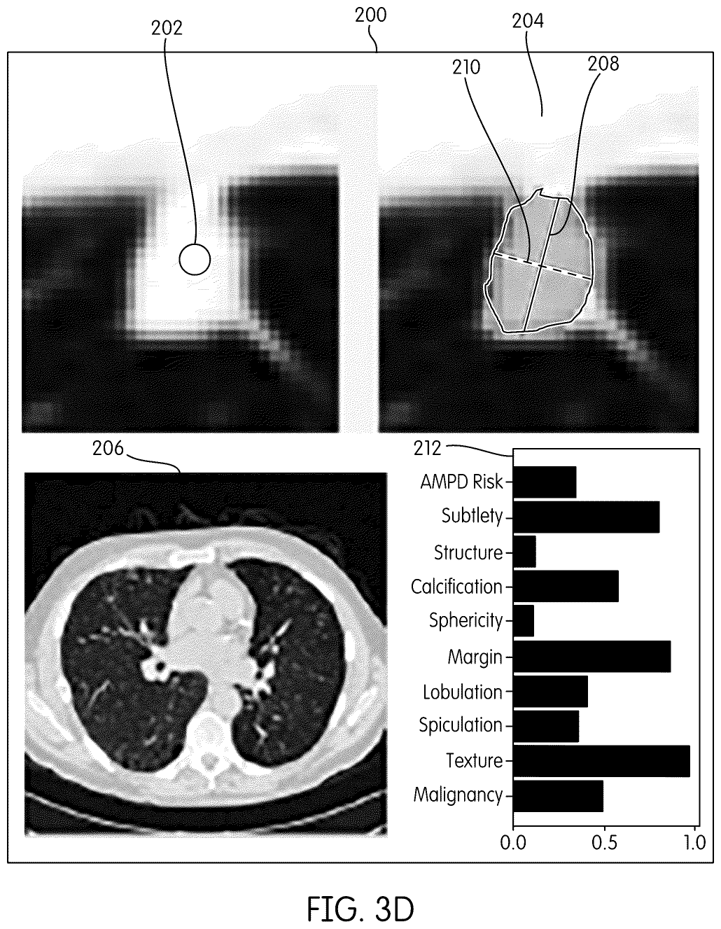

Frequently, the results of a method like method 10 will be displayed in a graphical user interface that is either separate from or integrated into another system, like a radiological information system. FIGS. 3A-3D are a series of illustrations of a graphical user interface (GUI) 200 illustrating the analytical results for a single tumor at several points in time. In each case, the interface 202 shows the indication of interest 202 (i.c., the point that was clicked), the segmentation/annotation of the lesion 204, and the original medical image 206. Also visible in the GUI 200 are major and minor axis measurements 208, 210, and a panel 212 that displays the phenotypic characteristics and risk scores.

FIGS. 3A-3D also illustrate that system 100 and other systems like it may be used to monitor a patient over time. In particular, FIG. 3A shows a malignant lung tumor one year before diagnosis, and FIG. 3B shows that same tumor in the year of diagnosis. Notably, the AMPD risk score displayed in the phenotypic characteristics panel 212 is high in both cases, higher in FIG. 3B than in FIG. 3A. By contrast, FIGS. 3C and 3D indicate the same benign lung nodule over several years of monitoring, with FIG. 3C showing an analysis of the nodule in year 1, and FIG. 3D showing an analysis of the nodule in year 2. In FIGS. 3C and 3D, the AMPD risk and malignancy scores are both considerably lower than with the malignant tumor of FIGS. 3A and 3B.

In general, method 10 and system 100 may be used to monitor a patient's progress, to confirm the efficacy of treatment, to predict the occurrence of side effects, or to monitor over time for recurrence. These are all potential “live clinical” uses. However, method 10 and system 100 may also be used to check the accuracy of diagnoses offered by human radiologists and oncologists, to search for evidence of malignancy before human eyes can find it, and for general research purposes.

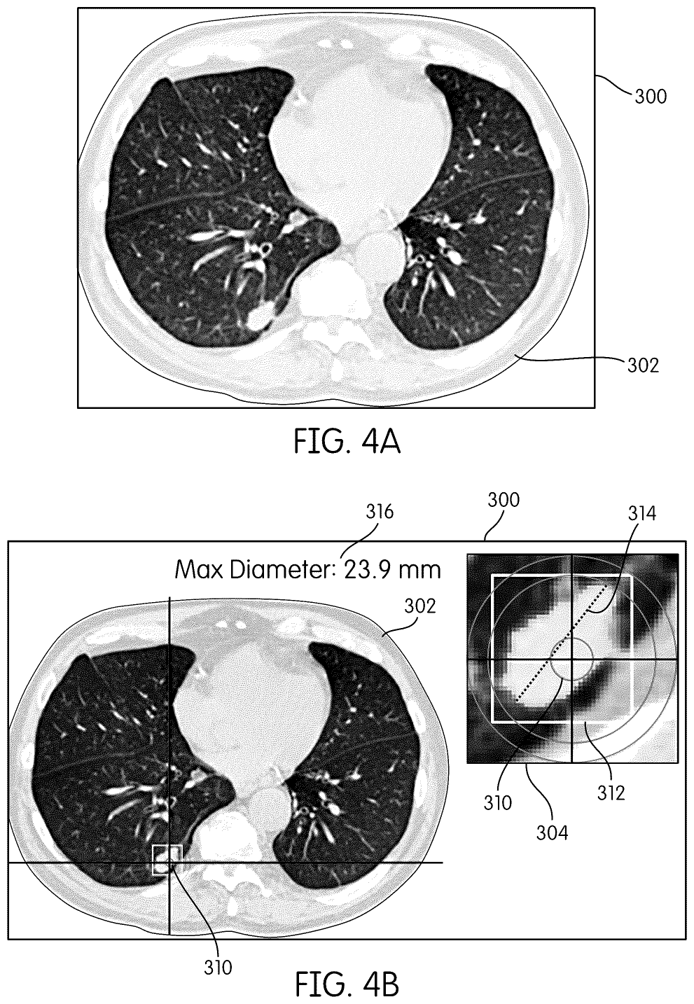

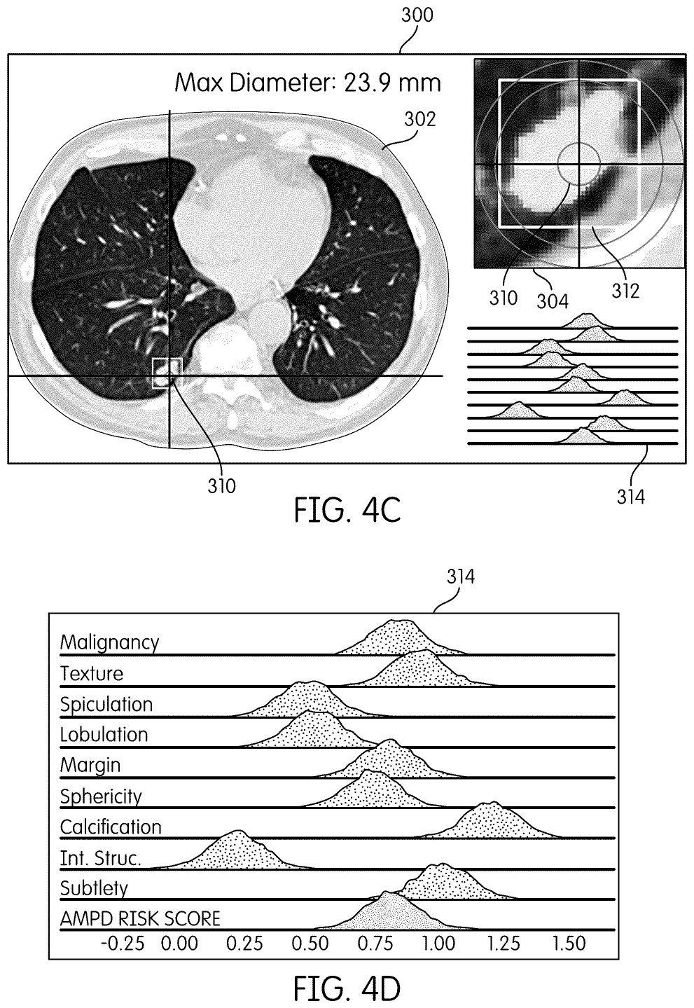

As may be clear from the above, method 10 and system 100 generate many different types of information. However, not all of that information need be presented at one time or in a particular interface. Moreover, in many cases, the interface will be dynamic, presenting contextually important information as needed. For example, as shown in FIG. 4A, the trained person may be viewing medical images in a GUI 300 that includes little more than the medical image 302 itself. When the trained person clicks on a lesion, the GUI 300 changes as shown in FIG. 4B, showing the indication of interest 310 (i.c., the point of click), and adding an enlargement panel 304 that shows an enlarged view of the indication of interest 310, the area 312 around the indication of interest that was segmented, and in this case, the major axis or maximum diameter of the lesion 314. In this case, the GUI 300 also indicates the measurement of maximum diameter at 316. However, the GUI 300 in this embodiment does not specifically display information on phenotype or other information, although in other embodiments, the trained person might press a key or make a selection to display such information.

In FIG. 4C, the GUI 300 includes the information noted above, and also includes a panel 314 describing the phenotypic and risk scores for the lesion. The panel 314 presents this information differently, showing a range or confidence interval for each score. FIG. 4D is an enlargement of the panel 314.

Method 10 returns at 32.

The description of method 10 above assumes a single prediction based on a single two-dimensional segmentation of a lesion from a single medical image acquired at a single timepoint. However, methods according to embodiments of the invention are not limited to this. As was described briefly above, method 10 and other methods according to embodiments of the invention may set of medical images acquired at the same time or at different times. If method 10 and other methods are applied to sets of medical images acquired at different times, then these methods and systems may be used for longitudinal monitoring. That is, method 10 and other methods and systems like it may be used to track and determine a lesion's response to treatment over time. If a system according to an embodiment of the invention is used for longitudinal monitoring, it may take into account all available medical images of the lesion over time. As new scans are taken and new medical images become available, the methods and systems may present the same medical predictions, updated or revised to include the new data, they may offer medical predictions of a different type that are clinically and contextually appropriate, or they may offer a mix of updated original and new, contextually-appropriate medical predictions.

In some cases, the medical predictions that are offered may be based, at least in some part, on the measurements that are taken. For example, if the measurements made during the annotation and measurement steps indicate that the lesion has grown, then one of the medical predictions may concern whether or not that apparent growth is true progression or hyperprogression. Similarly, if the measurements indicate that the lesion has progressed, the methods and systems may offer one or more medical predictions concerning whether or not the lesion is likely to benefit from some alternative treatment.

The description of method 10 presents its tasks in a certain order for case in explanation. The tasks need not necessarily be performed in the described order. For example, once a segmentation of the medical image is established, multiple tasks may be performed essentially in parallel. Moreover, certain tasks are described as being performed by certain types of machine learning models, but the nature of the model used for any particular task may vary greatly from embodiment to embodiment.

In the above embodiment, the U-net 102 used to segment the medical image is a deep learning model, a type of CNN. Other types of neural networks, like vision transformers, could also be used. Segmentation models that do not rely on neural networks could also be used, including thresholding, active-contour, and region-growing approaches. If the segmentation model does not use deep learning, extracted radiomic features could be used for medical prediction instead of features extracted from a deep learning model.

Deep learning could be used to generate more, or even all, of the necessary measurements and predictions. For example, the measurement model 134, phenotyping model 136, and risk score model 138 could all be deep learning models, like fully-connected CNNs. If deep learning models are used for these components 134, 136, 138, it may be necessary to encode the deep features from the U-net 102 in forms that the other deep learning models can use. For example, image-based deep features may be encoded in a vector form for input to deep learning models that do not take image input.

The use of multiple deep-learning machine models presents other opportunities as well. In the embodiment described above, the U-net 102 and any other machine learning models 134, 136, 138 would often be trained separately. However, it is possible to use multi-task learning to train multiple machine models at the same time. That is, cach machine learning model 102, 134, 136, 138 has a loss function associated with it. It is possible, and in some embodiments, it may be desirable, to train several machine learning models at once, i.c., to simultaneously optimize more than one loss function.

As those of skill in the art will understand, machine models must be trained before they can provide segmentations, predictions, and other kinds of useful output. Various training techniques could be used. Training the machine learning models 102, 134, 136, 138 described here would typically use a dataset of medical images of the desired type (CT, MRI, PET, etc.) with pathologically-confirmed diagnoses. For example, data from The Lung Image Database Consortium (LIDC) may be suitable for at least some uses, and the private image collections of hospitals or radiology practices could be used in other cases. The images should be sufficiently numerous and diverse in patient demographics, lesion type, phenotypical characteristics, and outcomes as to give the machine learning models 102, 134, 136, 138 sufficient exposure to a variety of different types of situations. In a typical arrangement, the available training data would be divided into two cohorts: a first cohort of data would be used for initial training, and a second cohort of data would be used for validation prior to deployment of any system 100. If necessary, the models 102, 134, 136, 138 could be retrained with adjustments until some defined performance metric is met, such as the area under the curve (AUC) of the receiver operating characteristic curve (ROC) for the model 102, 134, 136, 138.

In this description, unless the term “model” is qualified in such a way as to indicate its nature (e.g., a machine learning model, deep learning model, etc.), the term should be interpreted more broadly. For example, a nomogram is a type of model that may be used in and with embodiments of the invention. Nomograms may be used with the medical predictions generated by method 10 and output as a part of descriptive clinical reports or in other ways. A nomogram might, for example, be used to compare or combine the predictions from two or more machine learning models. A simple, non-trainable combination strategy leveraging features of import should also be considered a model. For instance, the averaging or summing of several key features to derive an aggregate score would be an example of a simple model.

Any of the methods described here may be implemented as a set of sets of machine-readable instructions on a machine-readable medium that, when executed, cause the machine to perform the method. The methods may also be implemented as a system of interconnected computing components. For example, the GUI used by the trained person to obtain the indication of interest may be a local computing device, while the computers or devices used to perform the other computations described here may be remote or “cloud-based” machines that are connected by a network, such as the Internet. Some or all portions of methods according to embodiments of the invention may also be performed by embedded systems. For example, the capability of creating an appropriate image segmentation of a medical image may be built into a medical imaging device or another such machine.

As those of skill in the art will appreciate, the tasks of a method like method 10 need not all happen in a continuous sequence, and in some cases, certain or all tasks may be fully automated. For example, all medical images described in metadata as being of a particular type (e.g., lung CT images) may be automatically segmented on entry to a PACS database, or shortly thereafter, with the remainder of the tasks of method 10 performed only on demand by a trained person. In yet other embodiments, methods like method 10 may be run in fully automated fashion, with images automatically segmented, lesions automatically identified, measurements and phenotypical characteristics established, and predictions made without an explicit indication of interest from a trained person. In those cases, a finished report on any identified lesions may simply be sent to, and saved in, an EHR system.

While several different forms of GUI output are illustrated in the figures, the output used in any particular method or system according to an embodiment of the invention may differ. Not all embodiments or implementations need use all of the output generated by method 10 and methods like it. As one example, the output of method 10 may be used to create a pop-up alert in an EHR system that a particular patient is unlikely to respond to a particular treatment, like an immune checkpoint inhibitor. Any treatment or drug recommendations may be used as alerts, prompts, or guidelines in a computer physician order entry system or in a pharmacy information system.

### EXAMPLE

An implementation of a single-click annotation, measurement, phenotyping, and diagnosis (AMPD) system was constructed for lung cancer screening. The AMPD system was validated on 3,073 nodules from 1,394 patient CT scans.

Methods and Materials: AMPD included two components: a click based annotation and measurement (AM) module and a deep phenotyping diagnosis (PD) module. AM was a click-based, pan-cancer deep learning segmentation model trained with 32,735 lesions from 4,427 patients from the DeepLesion dataset. The AM module was validated on screening CTs of 851 patients with 2530 nodules from the Lung Image Database Consortium (LIDC) dataset. Nodules were annotated and measured by 4 radiologists, as well as rated with respect to 9 phenotypic properties: suspicion of malignancy, texture, spiculation, lobulation, margin definition, sphericity, calcification, internal structure, and subtlety. A multitask PD model was trained to predict phenotypic attributes and overall diagnosis using deep features from the AM model in this dataset. The approach was evaluated end-to-end on a subset of the longitudinal National Lung Screening Trial (NLST) study of patients who had nodules >4 mm present in their first screening exam—94 were diagnosed with lung cancer on a subsequent CT scan, while 152 had stable nodules (<1.5 mm growth between exams) for 3 consecutive years. We Diagnostic performance was assessed at time of diagnosis and one year prior. Clicks were simulated by selecting a random point within the middle 50% of a radiologist-defined lesion annotation (LIDC) or bounding box (DeepLesion, NLST).

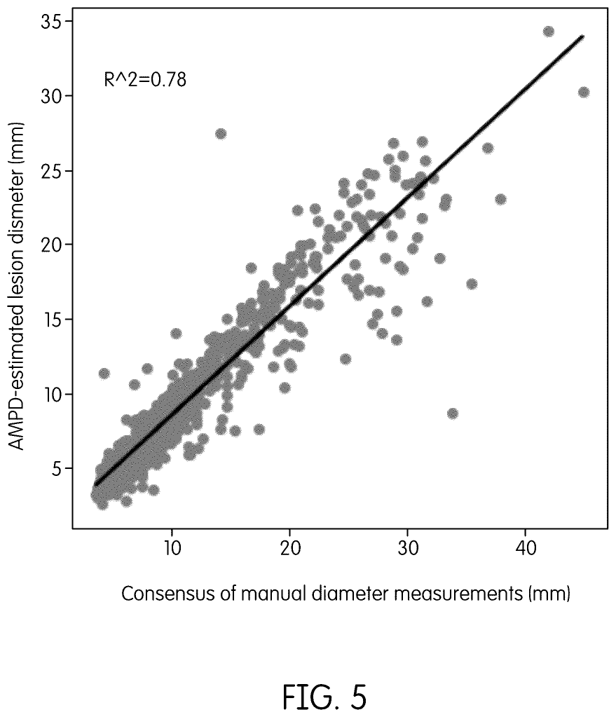

Results: Within LIDC, the AM module showed strong alignment with consensus of manual radiologist annotations (dice=0.81) and diameter measurements (intraclass correlation coefficient=0.86, p<1e-10, measurement error=1.20+/−2.99 mm). Deep feature phenotype scores from the PD model were significantly correlated with radiologist ratings (spearman correlation=0.06-0.62, p <=0.004). Leveraging this information, the PD model strongly predicted malignancy both at time of diagnosis (AUC=0.81) and one year prior (AUC=0.73). Lesion diameter was less predictive than PD (AUC-0.79 and AUC-0.68, respectively) and did not improve its performance when added to the model (AUC=0.83, AUC-0.74). FIG. 5 is a graph showing the consensus of manual lesion diameter measurements with AMPD-estimated lesion diameter. Intra-class correlation with radiologist consensus was 0.87 (p<1e-10), comparing favorably with human readers (ICC=0.93, p<1e-10), and average measurement error was 1.20+/−2.99 mm.

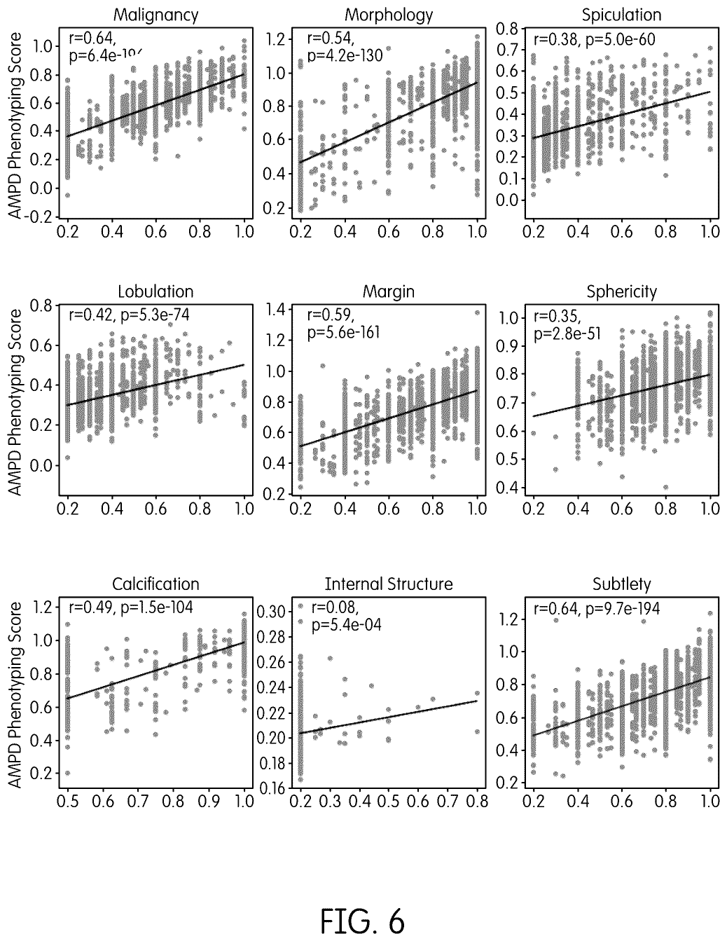

FIG. 6 is a set of graphs illustrating the consensus between manual measurements of nine phenotypical characteristics: malignancy, morphology, spiculation, lobulation, margin, sphericity, calcification, internal structure, and subtlety. As is illustrated, model-generated phenotype scores were significantly correlated with radiologist consensus ratings (e.g., spearman correlation=0.06-0.62, p<=0.004).

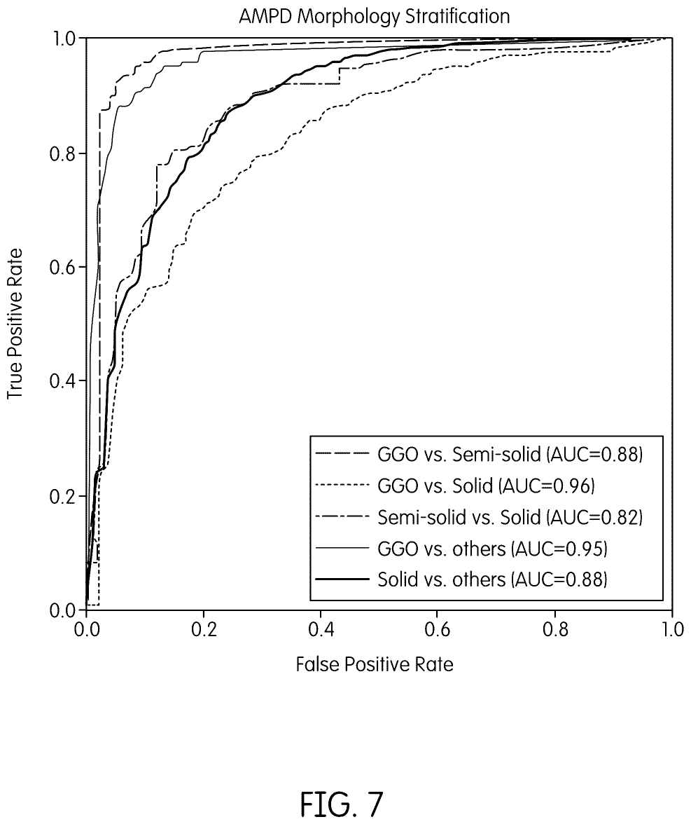

FIG. 7 is a graph illustrating the receiver-operating curve (ROC) for AMPD morphology stratification. The graph illustrates an assessment of whether the disclosed phenotyping module's morphology score could distinguish clinically significant morphologic groups. For the assessment, patients were grouped into most common diagnostic categories (solid, semi-solid, ground-glass opacity (“GGO”)), and a subset (1,145 nodules, 468 patients) where readers agreed on morphology was used to assess performance.

From a single click, the described implementation of the AMPD system produced high quality annotations and measurements that strongly aligned with the consensus of expert readers, and, furthermore, generated interpretable diagnostic predictions that can predate a clinical finding of malignancy. Following additional prospective multi-site validation, it has been determined that implementations of an AMPD system could both streamline traditional lung screening protocols (e.g., Lung-RAD v1.1) and identify malignancy sooner.

While the invention has been described with respect to certain embodiments, the description is intended to be exemplary, rather than limiting. Modifications and changes may be made within the scope of the invention, which is defined by the appended claims.

What is claimed is:

1. A method, comprising:
establishing one or more measurements and one or more phenotypical characteristics of a lesion using a first machine learning model operating on a machine segmentation of a lesion indicated in a medical image;
using the first machine learning model or a second machine learning model, providing a medical prediction concerning the lesion using one or more of the one or more measurements, the one or more phenotypical characteristics, or features extracted from the machine segmentation; and
outputting the medical prediction with at least one of the one or more measurements or at least one of the one or more phenotypical characteristics.

2. The method of claim 1, further comprising:
obtaining an indication of interest from a trained person and segmenting the medical image to create the machine segmentation based on the indication of interest.

3. The method of claim 2, wherein said segmenting comprises segmenting the medical image with a deep learning model trained to distinguish the lesion from other structures.

4. The method of claim 3, wherein the deep learning model comprises a U-net.

5. The method of claim 3, wherein said providing the medical prediction comprises using the features extracted from the machine segmentation.

6. The method of claim 5, wherein the features extracted from the machine segmentation comprise radiomic features.

7. The method of claim 5, wherein the features extracted from the machine segmentation comprise deep features extracted from the deep learning model.

8. The method of claim 2, wherein said obtaining comprises allowing the trained person to indicate a point or region of interest on the medical image using a graphical user interface and encoding that point or region of interest as the indication of interest.

9. The method of claim 1, wherein the one or more phenotypical characteristics comprise one or more of subtlety, structure, calcification, sphericity, margin, lobulation, spiculation, and texture.

10. The method of claim 1, wherein the one or more measurements comprise one or more of lesion diameter, short axis, area, volume, and conformity to a shape.

11. The method of claim 1, wherein the medical prediction comprises a diagnosis concerning the lesion; a classification of the lesion according to a phenotype or genotype;
a prediction of disease progression; a prediction of whether the lesion is likely to respond to a particular treatment; a prediction of whether an apparent growth of the lesion during a treatment represents a true progression or a pseudo-progression caused by the treatment; or a prediction of whether a particular patient is likely to experience a particular side effect.

12. The method of claim 1, wherein said providing the medical prediction uses the second machine learning model.

13. The method of claim 12, wherein the second machine learning model is a deep learning model.

14. The method of claim 13, wherein the first machine learning model and the second machine learning model are trained using multi-task learning.

15. The method of claim 1, wherein the medical prediction is a longitudinal medical prediction based on multiple medical images taken over time.

16. A method, comprising:
accepting an indication of interest indicating a point or region in a medical image using a graphical user interface;
based on the indication of interest, segmenting the medical image or a plurality of medical images for a single patient to identify a lesion in the medical image or the plurality of medical images using a segmentation machine learning model trained to identify the lesion;
extracting image features descriptive of the lesion from the medical image, the plurality of medical images, or the segmentation machine learning model;
establishing one or more measurements and one or more phenotypical characteristics of the lesion using one or more models;
generating a medical prediction concerning the lesion with a predictive machine learning model trained to use one or more of the image features descriptive of the lesion, the one or more measurements, or the one or more phenotypical characteristics; and
outputting the medical prediction, the one or more measurements, and the one or more phenotypical characteristics.

17. The method of claim 16, wherein the medical prediction is a longitudinal prediction.

18. The method of claim 16, wherein the medical prediction comprises a diagnosis concerning the lesion; a classification of the lesion according to a phenotype or genotype; a prediction of disease progression; a prediction of whether the lesion is likely to respond to a particular treatment; a prediction of whether an apparent growth of the lesion during a treatment represents a true progression or a pseudo-progression caused by the treatment; or a prediction of whether a particular patient is likely to experience a particular side effect.

19. The method of claim 16, wherein the segmentation machine learning model is a deep learning machine model and the one or more image features are deep features extracted from the deep learning machine model.

20. The method of claim 16, wherein the one or more image features are radiomic features.

21. A system, comprising:
a segmentation and annotation module having at least one segmentation machine learning model that accepts a medical image and an indication of interest, segments the medical image at least in a vicinity of the indication of interest to identify a lesion, the segmentation and annotation module producing annotations of the lesion using output of the at least one segmentation machine learning model; and
a phenotyping module having at least one phenotyping machine learning model that uses image features descriptive of the lesion in the medical image as input to produce measurements or scores for at least one phenotypical characteristic of the lesion.

22. The system of claim 21, wherein the image features are radiomic features extracted from the medical image one or both of within or around the lesion.

23. The system of claim 21, wherein the at least one segmentation machine learning model is a deep learning segmentation model and the image features are deep features extracted from the deep learning segmentation.
